(19) United States
(12) Patent Application Publication
(10) Pub. No.: US 2021/0262680 A1
(43) Pub. Date: Aug. 26, 2021

(54) PERSONAL ENVIRONMENTAL CONTROL SYSTEM AND METHOD

(71) Applicant: Carrier Corporation, Palm Beach Gardens, FL (US)

(72) Inventor: Nan Zhao, Cambridge, MA (US)

(21) Appl. No.: 17/248,327

(22) Filed: Jan. 20, 2021

Related U.S. Application Data
(60) Provisional application No. 62/981,230, filed on Feb. 25, 2020.

(51) Int. Cl. F24F 11/30; F24F 3/12; F24F 11/57; F24F 11/64; F24F 11/65; G05B 15/02; H05B 47/115; A61L 9/00; H04R 3/00; G06F 3/16; G06F 3/14

(52) U.S. Cl. CPC F24F 11/30; F24F 3/12; F24F 11/57; F24F 11/64; F24F 11/65; F24F 2003/003; H05B 47/115; A61L 9/00; H04R 3/00; G06F 3/165; G06F 3/14; G05B 15/02

(57) ABSTRACT

An environmental control system for controlling a personal environment at habitable space, the system including: a user occupied comfort subsystem having at least one of a user component, an illumination subsystem, and a microclimate subsystem, wherein the microclimate subsystem provides at least one of heating, cooling and ventilation to the personal environment; a sensory experience subsystem in network communication with the comfort subsystem; a controller in network communication with each of the comfort subsystem and the sensory experience subsystem, the controller configured to monitor and control at least one environmental parameter of the environmental control system.

### CROSS REFERENCE TO A RELATED APPLICATION

The application claims the benefit of U.S. Provisional Application No. 62/981,230 filed Feb. 25, 2020, the contents of which are hereby incorporated in their entirety.

### BACKGROUND

This disclosure generally relates to a personal environment, for instances homes, and private and public establishments relating to work, school, education and entertainment, where a person may engage in an activity, such as standing, sitting, resting or sleeping. In particular, this disclosure relates to a system and method for controlling a personal environment in a habitable space to enhance user productivity, comfort and well-being.

Habitable spaces may include indoor spaces or in some instances, outdoor spaces. For example, habitable spaces may include homes, office buildings, schools, libraries, restaurants, temporary accommodations (e.g., hotel, motels, resorts), public transportation facilities (e.g., airports, train stations), medical buildings and care facilities (e.g., hospitals, doctor's offices, rest homes), government buildings, leisure and entertainment facilities (e.g., clubs, spas, amusement parks, museums, theaters, arenas, casinos), public shopping places (e.g., shopping malls), and at other places where people may gather for work and non-working activities. Habitable spaces may be found on land, at sea or in the air, such as in private and commercial marine vessels (e.g., cruise ships), and private and commercial aircraft.

In general, a personal environment within a habitable space includes a place where a user may engage in an activity such as standing, sitting, resting or sleeping utilizing, for example, a desk or workspace, or a chair, sofa, recliner, lounge, or bed. As people spend more time engaged in such activities, there is a growing demand for greater control over personal space to optimize comfort and/or productivity. This includes the ability to control illumination, temperature, and sensory input to the personal environment which may be within the habitable space.

A habitable space typically has at least one lighting, heating and/or cooling solution. A heating and/or cooling solution may be a commercial or residential heating, ventilation and air conditioning (HVAC) system that has a central control, but which does not permit a person to directly control climate settings. For example, a hotel, or public library may have a central HVAC system to provide heating and/or cooling to a habitable space (e.g., a lobby, dining and lounge area, reading room). Visitors generally do not have permission or control to change climate settings in such spaces. One bothersome solution is to wear layered clothing or bring additional clothing in case the climate in the habitable space is uncomfortably cold. In this case, it would be preferable to have direct control over the climate. A similar problem exists with general lighting. Overhead ceiling lights, for example, may not provide sufficient light into a habitable space for certain activities, such as reading. It would be preferable to have control over lighting so that any activity may be enjoyed. Finally, with the advent of mobile phones, personal computers and tablets, a person may watch a video or listen to music through headphones or speakers; however, there may be little opportunity to enjoy a coordinated experience to enhance a personal environment (e.g., a personalized surround audio visual subsystem).

What is needed then, is a method and system that allows a person to control aspects of a personal environment such as illumination, heating, cooling, ventilation and sensory characteristics to enhance personal comfort and wellbeing.

### BRIEF DESCRIPTION

According to one non-limiting embodiment an environmental control system for controlling a personal environment in habitable space, the system including a user occupied comfort subsystem having at least one of a user component, an illumination subsystem, and a microclimate subsystem, wherein the microclimate subsystem provides at least one of heating, cooling and ventilation to the personal environment; a sensory experience subsystem in network communication with the comfort subsystem; and a controller in network communication with each of the comfort subsystem and the sensory experience subsystem, the controller configured to monitor and control at least one environmental parameter of the environmental control system.

In addition to one or more of the features described above, or as an alternative, in further embodiments, an environmental control system wherein the user component is at least one of a chair, sofa, recliner, couch, lounge, bench, work table, desk, bed, stationary bicycle.

In addition to one or more of the features described above, or as an alternative, in further embodiments, an environmental control system including an illumination subsystem having a lighting source for providing lighting to the personal environment.

In addition to one or more of the features described above, or as an alternative, in further embodiments, an environmental control system wherein the sensory experience subsystem includes at least one of a visual subsystem, audio subsystem, a speaker subsystem, a scenting subsystem.

In addition to one or more of the features described above, or as an alternative, in further embodiments, an environmental control system wherein the visual subsystem includes a device for delivery in pictorial form at least one of a video or image to the personal environment.

In addition to one or more of the features described above, or as an alternative, in further embodiments, an environmental control system wherein the audio subsystem includes a device for recording or reproducing sound for delivery to the personal environment.

In addition to one or more of the features described above, or as an alternative, in further embodiments, an environmental control system wherein the speaker subsystem includes a device for converting electrical signals into sound for delivery to the personal environment.

In addition to one or more of the features described above, or as an alternative, in further embodiments, an environmental control system wherein the scenting subsystem includes a device for providing at least one of a scent or fragrance for delivery to the personal environment.

In addition to one or more of the features described above, or as an alternative, in further embodiments, an environmental control system including the controller configured to initiate, adjust or terminate at least one environmental parameter relating to at least one of a user component, an illumination subsystem, a microclimate subsystem, an audio subsystem, a visual subsystem, a speaker subsystem and a scenting subsystem.

In addition to one or more of the features described above, or as an alternative, in further embodiments, an environmental control system, wherein an environmental parameter includes selecting, initiating, adjusting or terminating at least one of a level or range and pertaining to at least one of a: lighting source, luminance, lighting intensity, lighting distribution, lighting color temperature, sound source, sound distribution, sound selection, sound level, heating source, heating intensity, ventilation source, ventilation intensity, cooling source, cooling intensity, speaker source, speaker volume, image source, image selection, scent selection, scent intensity.

According to another non-limiting embodiment a method for establishing a personal environment in a habitable space using an environmental control system, the method including: determining whether the comfort subsystem having at least one of a user component, an illumination subsystem, and a microclimate subsystem, is occupied by a user; determining the status of at least one environmental parameter of least one of the comfort subsystem and the sensory experience subsystem within the personal environment; establishing a connection between a controller, and at least one of the comfort subsystem and the sensory experience subsystem; and operating a controller to change at least one environmental parameter of at least one of the comfort subsystem and the sensory experience subsystem.

In addition to one or more of the features described above, or as an alternative, in further embodiments, a method for establishing a personal environment in a habitable space using an environmental control system, the method wherein the user component is at least one of a chair, sofa, recliner, couch, lounge, bench, work table, desk, bed, stationary bicycle.

In addition to one or more of the features described above, or as an alternative, in further embodiments, a method for establishing a personal environment in a habitable space using an environmental control system, the method wherein the illumination subsystem comprises a lighting source for providing lighting to the personal environment.

In addition to one or more of the features described above, or as an alternative, in further embodiments, a method for establishing a personal environment in a habitable space using an environmental control system, the method wherein the sensory experience subsystem comprises at least one of a visual subsystem, audio subsystem, a speaker subsystem, a scenting subsystem.

In addition to one or more of the features described above, or as an alternative, in further embodiments, a method for establishing a personal environment in a habitable space using an environmental control system, the method wherein the visual subsystem comprises a device for delivery in pictorial form at least one of a video or image to the personal environment.

In addition to one or more of the features described above, or as an alternative, in further embodiments, a method for establishing a personal environment in a habitable space using an environmental control system, the method wherein the audio subsystem comprises a device for recording or reproducing sound for delivery to the personal environment.

In addition to one or more of the features described above, or as an alternative, in further embodiments, a method for establishing a personal environment in a habitable space using an environmental control system, the method wherein the speaker subsystem comprises a device for converting electrical signals into sound for delivery to the personal environment.

In addition to one or more of the features described above, or as an alternative, in further embodiments, a method for establishing a personal environment in a habitable space using an environmental control system, the method wherein the scenting subsystem comprises a device for providing at least one of a scent or fragrance for delivery to the personal environment.

In addition to one or more of the features described above, or as an alternative, in further embodiments, a method for establishing a personal environment in a habitable space using an environmental control system, the method including configuring the controller to initiate, adjust or terminate at least one environmental parameter relating to at least one of a user component, an illumination subsystem, a microclimate subsystem, an audio subsystem, a visual subsystem, a speaker subsystem and a scenting subsystem.

In addition to one or more of the features described above, or as an alternative, in further embodiments, a method for establishing a personal environment in a habitable space using an environmental control system, the method including configuring the controller to simultaneously deliver an environmental parameter based on at least one of a user profile to at least one of a user component, an illumination subsystem, a microclimate subsystem, an audio subsystem, a visual subsystem, a speaker subsystem and a scenting subsystem.

In addition to one or more of the features described above, or as an alternative, in further embodiments, a method for establishing a personal environment in a habitable space using an environmental control system, the method wherein an environmental parameter includes selecting, initiating, adjusting or terminating at least one of a level or range and pertaining to at least one of a: lighting source, luminance, lighting intensity, lighting distribution, lighting color temperature, sound source, sound distribution, sound selection, sound level, heating source, heating intensity, ventilation source, ventilation intensity, cooling source, cooling intensity, speaker source, speaker volume, image source, image selection, scent selection, scent intensity.

### BRIEF DESCRIPTION OF THE DRAWINGS

The accompanying drawings form a part of the specification. Throughout the drawings, like reference numbers identify like elements.

FIG. 1 is a graphical representation of an environmental control system in accordance with embodiments of the disclosure.

FIG. 2 is a method for establishing a personal environmental using an environmental control system in accordance with embodiments of the disclosure.

The detailed description explains embodiments of the present disclosure, together with advantages and features, by way of example with reference to the drawings.

### DETAILED DESCRIPTION

As will be described in greater detail below the present disclosure provides for an environmental control system 100 and method for controlling a personal environment in a habitable space. It should be evident however to one skilled in the art that the present disclosure is not limited to the specific examples given and could be used in other habitable spaces.

The environmental control system 100 utilizes at least one of a comfort subsystem 10, a sensory experience subsystem 20, a controller 30 and a server 40, in a direct or wireless communication network to provide an interactive experience using related environmental parameters, to create a desired environment (e.g., tropical) in a personal environment.

Comfort subsystem 10 may include one or more sub-subsystems, including a user component 12, an illumination subsystem 14 for controlling illumination to the personal environment and/or a microclimate subsystem (e.g., heating, cooling, ventilation) 16 for controlling temperature within the personal environment. Sensory experience subsystem 20 may also include one or more sub-subsystems for delivering a coordinated experience to a personal environment. The sensory experience subsystem 20 may include a visual subsystem 22 for providing an image (e.g., picture, video) to the personal environment; an audio subsystem 24 for providing a sound recording to the personal environment, a speaker subsystem 26 for delivering sound to the personal environment, and a scenting subsystem 28 for delivering a scent or fragrance 24 to the personal environment. Controller 30 may include, but is not limited to a control device (e.g., remote), a mobile device (e.g., phone, table, computer, etc.) or computer.

The environmental control system 100 network may include a computer server 40, for storing information relating to the environmental control system 100, including environmental parameters, and data received from and transmitted within an environmental control system 100. The server 40, may include a cloud computing system that is network connected anywhere in the world. Environmental control system 100 data communications may be carried out using any of a variety of custom or standard wired or wireless protocols for commercial or residential use (Wi-Fi, ZigBee, 6LoWPAN, CAT6 Ethernet, HomePlug, etc.).

The environmental control system 100 may include one or more sensors or devices that permits the comfort subsystem 10, the sensory experience subsystem 20, and each of their respective sub-subsystems, the controller 30, and server 40, to share and communicate data to provide a coordinated user experience, as further described below.

Turning to the comfort subsystem 10, a user component 12 may be any apparatus that permits a user to engage in an activity in the personal environment that may include, but is not limited to standing, sitting, resting or sleeping. A user component 12 may include any one or more of a chair, a sofa, a recliner, a couch, a lounge, a bench, a work table, a desk, a bed, a stationary bicycle.

Illumination subsystem 14 may include at least one of a lighting element (e.g., light bulb, LED, halogen) for providing illumination to the personal environment. The illumination subsystem may permit lighting to the personal environment to be adjustable, for example, lighting intensity, lighting distribution, lighting color or temperature.

Microclimate subsystem 16 may include at least one of a heating, cooling or ventilation assembly. The heating assembly may include at least one of an electric heating element for providing heat to the personal environment. The cooling assembly may include at least one of an air conditioning unit and/or fan for providing cooling and/or ventilation to the personal environment.

An environmental parameter relates to at least one of a: lighting source, luminance, lighting intensity, lighting distribution, lighting color temperature, sound source, sound distribution, sound selection, sound level, heating source, heating intensity, ventilation source, ventilation intensity, cooling source, cooling intensity, speaker source, speaker volume, image source, image selection, scent selection, scent intensity. The environmental control system 100 may be configured to store at least one environmental parameter. A controller 30 may be configured to retrieve a stored environmental parameter, or to initiate (turn on), terminate (turn off) or adjust an environmental parameter. An environmental parameter may be a value. For example, setting a sound volume within a range of 0 decibels to 20 decibels; or may be a range.

An environmental parameter may include data provided by a user. For example, a user may select a preferred environmental temperature range of 72-75° F. In some embodiments, an environmental parameter may be provided or adjusted, by feedback obtained from a sensor or device within the environmental control system 100 subsystem, as discussed below.

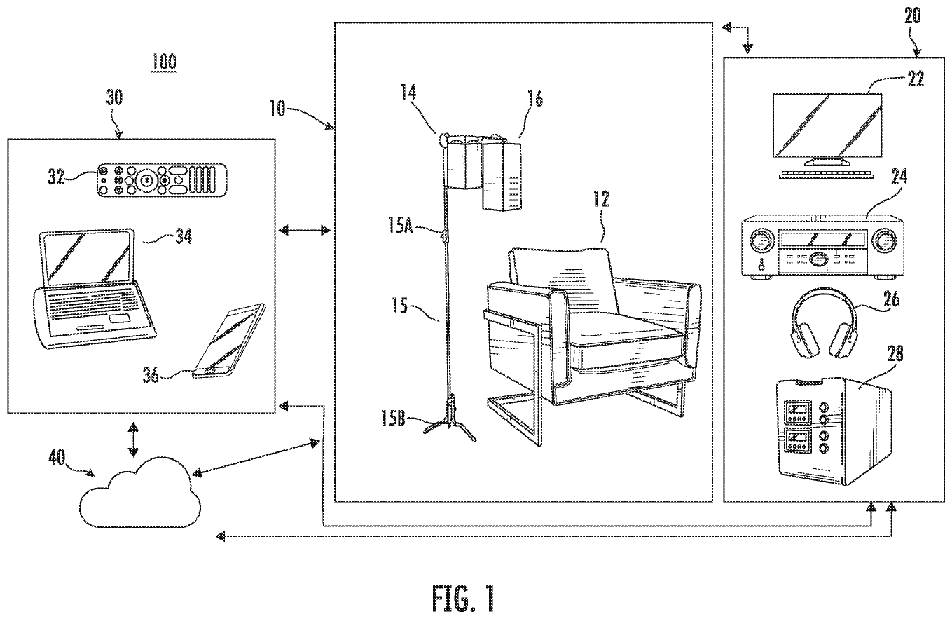

FIG. 1 illustrates the illumination subsystem 14 and microclimate subsystem 16, operably coupled in a housing. The illumination subsystem 14 and microclimate subsystem 16, may each have different dimensions, and may be in a configuration wherein the microclimate system 16 is distal to member 15. FIG. 1 is provided for illustrative purposes only. The illumination subsystem 14 and microclimate subsystem 16 may be separate assemblies, independently housed, or may be combined in a unitary housing.

FIG. 1 shows illumination subsystem 14 and microclimate subsystem 16 separate from user component 12, and the sensory experience subsystem 20. However, the subsystems and components of environmental control system 100 may be operably coupled to or integrated with any other subsystem or component in any combination. By way of example, and not limitation, user component 12 may have integrated therein, a lighting element and a fan.

The illumination subsystem 14 and microclimate subsystem 16 may be mounted on member 15 in a configuration that permits one or both to be detached from member 15, and replaced, if necessary. In an alternate embodiment, at least one of the illumination subsystem 14 and microclimate subsystem 16 may attached to member 15 by way of at least one of a coupler and fastener. The illumination subsystem 14 and microclimate subsystem 16 may be vertically movable along member 15 by a mechanical or automated means.

Member 15 may be substantially vertical of a predetermined non-adjustable height. Member 15 may be a unitary member (e.g., a post) or may have multiple parts that allow for height adjustment (e.g., nested collapsible members which lock in place by a locking mechanism). Member 15 may have one or more connections that allow member 15 to adjust in any direction to change the vertical or horizontal orientation of at least one of the illumination subsystem 14 and the microclimate subsystem 16. For example, member 15 may have a joint 15A that allows a user to bend member 15 (e.g., 30°) toward the user component 12 to direct more ventilation to user component 12.

Visual subsystem 22 may be any device for providing information in pictorial form (e.g., image or video). For example, the visual subsystem may be a monitor to which may be connected to any device having access to or stored therein, at least one of an image or video. For example, a monitor may be directly or wirelessly connectable to one or more computers, tablets, mobile devices, storage devices (e.g., USB flash drive), and interface devices (e.g., Apple® TV, Amazon® Fire TV stick). Trademarks are the property of their respective owners.

Audio subsystem 24 may be any electronic device for recording or reproducing sound. The audio subsystem may include but is not limited to a radio, phonograph, CD/DVD player, tape deck, a device capable of playing digitally recorded music in at least one audio file format including compressed or uncompressed digital audio files (e.g., MP3/MP4, WAV, AIFF, AU, PCM, FLAC, AAC, OGG, WMA).

Speaker subsystem 26 may be any electro-acoustical device that converts electrical signals into sound. The speaker subsystem 26 may include, but is not limited to headphones, earphones, and free-standing speaker devices for delivering at least one of a sound to a personal environment.

Scenting subsystem 28 may be any device for providing at least one of a scent or fragrance to a personal environment.

The environmental control system 100 may include components such as integrated circuits, conductors, transducers, optical devices, sensors, devices for supplying power, microcontrollers, microprocessors and memory (volatile and/or non-volatile), and/or components for one-way or two-way wired or wireless communication (e.g., transmitter/receiver, antennae, RFID technology) between and among the environmental control system 10, the comfort subsystem 10, the sensory experience subsystem 20, their respective sub-systems, controller 30, or server 40. These components may be used to provide input into controller 30, and/or to receive biometric feedback from, the environmental control system 100 to provide a coordinated personal environment. These components may be attached to or operably coupled to any one or more of the comfort subsystem 10, the sensory experience subsystem 20, their respective sub-systems, and/or controller 30, for example by a wire assembly, or wireless communication.

In one non-limiting embodiment, the environmental control system 100 may have disposed therein at least one of an energy-transducing member, electromagnetic energy sensor, light energy sensor and/or a light emitting member, thermal energy or temperature sensor. Other non-limiting examples of energy-transducing members may include inertial sensor, inclinometer, vibration sensor, motion-based user interface, gesture-based user interface, bend sensor, pressure sensor, air pressure sensor, airflow sensor, altimeter, micro electromechanical system (MEMS) actuator, piezoelectric actuator, tactile-sensation-creating member, tactile user interface, inflatable member, touch screen, touch-based human-to-computer textile interface, touchpad, virtual projected keypad, and gesture recognition sensor, global positioning system (GPS) module, and compass. To illustrate, the user component 12 may have a heating element for providing heat to a user occupying the user component 12 when the sensory experience subsystem is also providing related images, sounds and scents for a personal experience. In this example, the environmental control system 100 may be configured to have stored therein at least one themed personal experience, for example, “ski-lodge.” The themed experience may be stored on the controller 30 or on a server 40. When the environmental control system 100 is activated, the comfort subsystem 20 and the sensory experience subsystem 20 are also activated. Once activated, the environmental control system 100 may be configured to communicate with each of the comfort subsystem 10 and the sensory experience subsystem 20 to adjust at least one environmental parameter of a comfort subsystem 20 and/or the sensory experience subsystem 20.

The characteristics of an environmental parameter (e.g., type, quality, intensity) of one subsystem may be similar to the characteristics of another environmental parameter to provide a coordinated environmental control system 100 response. To illustrate, when the environmental control system 100 is activated the environmental control system 100 may adjust an environmental parameter in user component 12 to increase the amount of heat delivered from a chair to a seated user. The microclimate subsystem 16 may also at the same time, adjust an environmental parameter to provide an increased level of heat from a heating element disposed. The visual subsystem 22 may also adjust an environmental parameter to turn on and deliver images of logs burning in a fireplace. The audio subsystem 24 and the speaker subsystem 26 may also adjust environmental parameters to deliver and increase volume so that the sound of logs burning may be heard. The scenting system 28 may adjust an environmental parameter to deliver a light intensity “forest” scent.

Environmental control system 100 may also include an electromagnetic energy sensor, for example, a heart rate monitor, pulse rate monitor or breathing monitor. An electromagnetic energy sensor may provide input to controller 30 pertaining to an environmental selection or environmental preference. In one non-limiting embodiment, the controller 30 may be configured to activate, adjust or terminate the environmental control system 100, or any subsystem or sub-subsystem in response to input from a user or in response to feedback obtained from a subsystem. For example, the controller 30 may be configured to receive heart rate signals from the user component 12, or from a user device such as a smart watch. Upon receiving the signal, controller 30 may be further configured to also adjust all environmental parameters to a “0” intensity, thereby turning off the illumination subsystem 14, and the visual system 22, the audio subsystem 24 and speaker subsystem 26, and adjusting the environmental parameter of the scenting system 28 to provide a scent that a user may find soothing.

Environmental control system 100 may also include a light energy sensor and/or a light emitting member. In one non-limiting embodiment, a light intensity sensor may be used to detect current environmental lighting conditions, and/or may be used to sense light intensity within the habitable space. Other non-limiting examples of a light energy sensor may include, ambient light sensor, electro-optical sensor, infrared sensor, laser sensor, optical sensor, optoelectronic sensor, photoelectric sensor. For example, a light emitting diode integrated into the user component 12 may detect light intensity. Controller 30 may be configured to adjust an environmental parameter of the illumination subsystem 14 to change light intensity, focus, direction, etc., to the personal environment as lighting changes throughout the day or night. As the lighting in the personal environment changes over time, the environmental control system 100 may, for example, also deliver images, sounds and scents that are related to this change. In this example, as the lighting changes, the controller 30 may be configured to also adjust the environmental parameters of the illumination subsystem 14 to lower light intensity, the also activate the visual subsystem 22 to deliver images of food. The audio subsystem 24 and speaker system 26 may also deliver sounds that may be heard in a restaurant (e.g., clinking glasses, soft laughter, soft music).

Environmental control system 100 may also include a sound sensor, sensor, microphone, speech and/or voice recognition interface, ambient sound sensor or monitor, audiometer, sound-emitting member, speaker, audio speaker, sound masking or cancelling member, white or pink noise emitter, ultrasound emitter. For example, controller 30 may be configured to receive one or more voice commands for controlling the environmental control system 100 for selecting an environmental experience. For example, a user may command controller 30 to deliver a “tropical experience.” In this example, the controller 30 may activate the microclimate subsystem 16 and adjust environmental parameters to provide a “breeze” from a fan element, and/or provide heat to the user component 12. The visual system 22 may also at the same time, adjust environmental parameters of the other sub-subsystems to deliver images of tropical birds, and related sounds (e.g., waves crashing on a beach), and the scenting subsystem 28 delivers scents that may evoke images of the beach and ocean.

Environmental control system 100 may also include a thermal energy or temperature sensor. A thermal energy or temperature sensor device may include, a thermistor, thermometer, body temperature sensor, skin temperature sensor, ambient temperature sensor, heat pump, ambient air monitor. For example, an ambient temperature sensor integral or attached to the user component 12 may determine that the personal environment has an ambient temperature of 71° F. The controller 30 may be configured to store at least one user profile, wherein a user may store a setting for at least one environmental parameter (e.g., preferred ambient temperature of 76°). In one non-limiting embodiment, the controller 30 may detect the presence of a user in the personal environment, and automatically adjusts an environmental parameter within the user component 12 (e.g., heating element) to warm the user to a preferred temperature based on the user profile. The controller 30 may be further configured to also activate and adjust the environmental parameter of a heating element in the microclimate subsystem 16 to assist in warming the user.

In some embodiments, the user may have a profile stored on a controller 30 or server 40 that may be in communication with the environmental control system 100. A user profile may include general information about the user that may be relevant to the environmental experiences that may be available to a user in the personal environment. For example, environmental parameters for one or more subsystems, user age, weight, height, GPS location, place of residence, etc., as they may relate to the type of experience within a personal environment that a user may prefer or wish to implement. When the user approaches or is within the personal environment, the user's presence may be detected by the environmental control system 100 using for example, motion sensors within the personal environment, or by GPS or geolocation data which may be available in from a user's mobile device, such as a computer, phone, watch or tablet. In this example, as a user approaches the personal environment, his or her profile, including stored environmental parameters are communicated to and implemented by the environmental control system 100. In this example, a user steps into an active personal environment that is tailored to his or her profile.

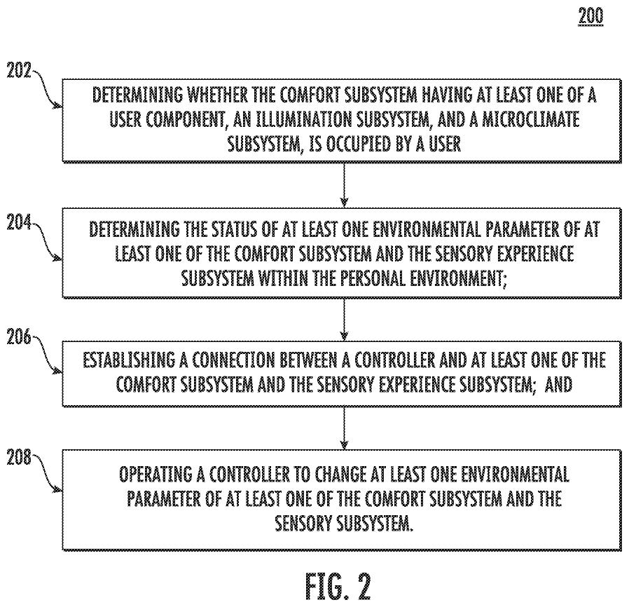

FIG. 2 shows a flow diagram disclosing an embodiment of a method for controlling a personal environment in a habitable space using an environmental control system, the method 200 including, determining whether the comfort subsystem having at least one of a user component, an illumination subsystem, and a microclimate subsystem, is occupied by a user; determining the status of at least one environmental parameter of least one of the comfort subsystem and the sensory experience subsystem within the personal environment; establishing a connection between a controller, and at least one of the comfort subsystem and the sensory experience subsystem; operating a controller to change at least one environmental parameter of at least one of the comfort subsystem and the sensory experience subsystem.

At the beginning of method 202 the environmental control system 100 includes a comfort subsystem, having at least one of a user component 12, an illumination subsystem 14 and a microclimate subsystem 16. The controller 30 determines that a user is occupying the comfort subsystem. For example, mobile devices may have location-aware capabilities that can receive location data from the mobile device directly, or a telecommunications carrier. To illustrate, the environmental control system 100 may be in communication with the controller 30, which may be a mobile device. When a user enters the personal environment space, the environmental control system 100 may turn on and become active. In another example, the environmental control system 100 may determine that a user is occupying a user component 12, such as when the user component sensors detect the presence of a user in a chair (e.g., pressure sensor). The method then proceeds to step 204.

Once the environmental control system 100 has been activated, it may determine at least one environmental parameter in step 204. For example, upon activation a subsystem may determine the current temperature within the personal environment. As discussed above, a user component 12 may have an ambient air temperature sensor. In another example, an audio subsystem may determine that the ambient noise level in the personal environment is above a predetermined limit. The method then proceeds to the next step.

In step 206, the environmental control system 100 establishes a connection between at least one of a controller 30, a server 40, and a subsystem. Once a connection has been established, the experience delivered to the personal environment may change based on at least at least one environmental parameter from at least one of a user or feedback from the environmental control system 100.

In step 208, environmental control system 100 operates to change at least one of an environmental parameter of at least one subsystem and sub-subsystem. In one non-limiting embodiment, environmental control system 100 may be configured to coordinated environmental parameter output to deliver a personal environment having related characteristics. For example, the environmental control system 100 may have a program for delivering a “tropical” experience.

To illustrate, the environmental control system 100 when activated, coordinates environmental parameter output to provide a coordinated “tropical” experience, such as turning on a heating element in a chair or microclimate device, adjusting lighting in the personal environment, delivering images of a tropical location, delivering sounds (e.g., calypso music, sounds of waves on a beach), and fragrances (e.g., fruits, flowers, etc.) associated with a tropical environment.

By way of example, an environmental parameter may include the light intensity of the illumination subsystem 14 based on a range of settings. For example, a setting of 0 may be off and a setting of 10 may be brightest light intensity. To illustrate, upon entering the personal environment, a light intensity setting may be changed within a range of light intensities using input from a user into controller 30. In some non-limiting embodiments, light intensity may be automatically changed by the environmental control system 100 based, for example based on data received from a sensor (e.g., ambient light sensors) or a device (e.g., mobile phone relating to time of day) and then adjusting light intensity to account for the of, or presence or absence of ambient light.

In yet another non-limiting embodiment, the parameter may be changed based on data stored in the controller 30 or a server 40 related to a user profile. For example, an ambient temperature sensor integral or attached to the user component 12 may determine that the personal environment has an ambient temperature of 71° F. The controller 30 may be configured to store at least one user profile, wherein a user may store a setting for at least one environmental parameter (e.g., preferred ambient temperature of 76°). The controller 30 may detect the presence of a user in the personal environment, and activate a heating element in the user component 12 to warm the personal environment to a user preferred temperature. The controller 30 may be further configured to also activate a heating element in the microclimate subsystem 16 to assist in warming the user while the sensory experience subsystem delivers at least one of a sound, image or fragrance providing a coordinated experience to the personal environment.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

We claim:

1. An environmental control system for controlling a personal environment in habitable space, the system comprising:
a user occupied comfort subsystem having at least one of a user component, an illumination subsystem, and a microclimate subsystem, wherein the microclimate subsystem provides at least one of heating, cooling and ventilation to the personal environment;
a sensory experience subsystem in network communication with the comfort subsystem; and
a controller in network communication with each of the comfort subsystem and the sensory experience subsystem, the controller configured to monitor and control at least one environmental parameter of the environmental control system.

2. The environmental control system of claim 1, comprising an illumination subsystem having a lighting source for providing lighting to the personal environment.

3. The environmental control system of claim 1, wherein the sensory experience subsystem comprises at least one of a visual subsystem, audio subsystem, a speaker subsystem, a scenting subsystem.

4. The environmental control system of claim 3, wherein the visual subsystem comprises a device for delivery in pictorial form at least one of a video or image to the personal environment.

5. The environmental control system of claim 3, wherein the audio subsystem comprises a device for recording or reproducing sound for delivery to the personal environment.

6. The environmental control system of claim 3, wherein the speaker subsystem comprises a device for converting electrical signals into sound for delivery to the personal environment.

7. The environmental control system of claim 3, wherein the scenting subsystem comprises a device for providing at least one of a scent or fragrance for delivery to the personal environment.

8. The environmental control system of claim 1, the controller configured to initiate, adjust or terminate at least one environmental parameter relating to at least one of a user component, an illumination subsystem, a microclimate subsystem, an audio subsystem, a visual subsystem, a speaker subsystem and a scenting subsystem.

9. The environmental control system of claim 1, the controller configured to simultaneously deliver an environmental parameter based on at least one of a user profile to at least one of a user component, an illumination subsystem, a microclimate subsystem, an audio subsystem, a visual subsystem, a speaker subsystem and a scenting subsystem.

10. The environmental control system of claim 1, wherein an environmental parameter includes selecting, initiating, adjusting or terminating at least one of a level or range and pertaining to at least one of a: lighting source, luminance, lighting intensity, lighting distribution, lighting color temperature, sound source, sound distribution, sound selection, sound level, heating source, heating intensity, ventilation source, ventilation intensity, cooling source, cooling intensity, speaker source, speaker volume, image source, image selection, scent selection, scent intensity.

11. A method for establishing a personal environment in a habitable space using an environmental control system, the method comprising, determining whether the comfort subsystem having at least one of a user component, an illumination subsystem, and a microclimate subsystem, is occupied by a user;
determining the status of at least one environmental parameter of least one of the comfort subsystem and the sensory experience subsystem within the personal environment;
establishing a connection between a controller, and at least one of the comfort subsystem and the sensory experience subsystem; and
operating a controller to change at least one environmental parameter of at least one of the comfort subsystem and the sensory experience subsystem.

12. The method of claim 11, wherein the illumination subsystem comprises a lighting source for providing lighting to the personal environment.

13. The method of claim 11, wherein the sensory experience subsystem comprises at least one of a visual subsystem, audio subsystem, a speaker subsystem, a scenting subsystem.

14. The method of claim 13, wherein the visual subsystem comprises a device for delivery in pictorial form at least one of a video or image to the personal environment.

15. The method of claim 13, wherein the audio subsystem comprises a device for recording or reproducing sound for delivery to the personal environment.

16. The method of claim 13, wherein the speaker subsystem comprises a device for converting electrical signals into sound for delivery to the personal environment.

17. The method of claim 13, wherein the scenting subsystem comprises a device for providing at least one of a scent or fragrance for delivery to the personal environment.

18. The method of claim 11, including configuring the controller to initiate, adjust or terminate at least one environmental parameter relating to at least one of a user component, an illumination subsystem, a microclimate subsystem, an audio subsystem, a visual subsystem, a speaker subsystem and a scenting subsystem.

19. The method of claim 11, including configuring the controller to simultaneously deliver an environmental parameter based on at least one of a user profile to at least one of a user component, an illumination subsystem, a microclimate subsystem, an audio subsystem, a visual subsystem, a speaker subsystem and a scenting subsystem.

20. The method of claim 11, wherein an environmental parameter includes selecting, initiating, adjusting or terminating at least one of a level or range and pertaining to at least one of a: lighting source, luminance, lighting intensity, lighting distribution, lighting color temperature, sound source, sound distribution, sound selection, sound level, heating source, heating intensity, ventilation source, ventilation intensity, cooling source, cooling intensity, speaker source, speaker volume, image source, image selection, scent selection, scent intensity.
